(19) United States
(12) Patent Application Publication
(10) Pub. No.: US 2005/0169856 A1
(43) Pub. Date: Aug. 4, 2005

(54) Coloring composition, process of making, uses thereof

(75) Inventor: Jean Francois Grollier, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/039,781

(22) Filed: Jan. 24, 2005

Related U.S. Application Data
(60) Provisional application No. 60/543,541, filed on Feb. 12, 2004.

(30) Foreign Application Priority Data
Jan. 29, 2004 (FR) 0400857

(51) Int. Cl. A61K 7/42; A61K 7/04; A61K 7/021

(52) U.S. Cl. 424/59; 424/63; 424/61

(57) ABSTRACT

The invention concerns a process for preparation of a composition for the permanent or semi-permanent coloring of the skin, the eyelids, the lips or the nails, where a fluid is percolated under a pressure of at least 3 bars through at least one coloring agent in solid or paste form, and the composition so made. It also concerns a process for the permanent or semi-permanent coloring of the skin, the eyelids, the lips or the nails using the coloring composition obtained according to the process of the invention.

### REFERENCE TO PRIOR APPLICATIONS

This application claims priority to U.S. provisional application 60/543,541 filed Feb. 12, 2004, and to French patent application 0400857 filed Jan. 29, 2004, both incorporated herein by reference.

### FIELD OF THE INVENTION

The present invention is directed to compositions useful for example for the permanent or semi-permanent coloring of the skin, the eyelids, the lips or the nails, etc., to a process for making such compositions, and to the permanent or semi-permanent coloring of keratin materials and/or mucous membranes such as the skin, the eyelids, the lips or the nails, etc., using the invention composition.

More particularly, the invention concerns a process for preparation of a composition for the permanent or semi-permanent coloring of the skin, the eyelids, the lips or the nails, where a fluid is percolated under a pressure of at least 3 bars through at least one coloring agent in solid or paste form, and the composition so made. It also concerns a process for the permanent or semi-permanent coloring of the skin, the eyelids, the lips or the nails using the coloring composition obtained according to the process of the invention.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Typical cosmetic products such as for example blushers, eye shadow, foundation creams, concealers or lipsticks generally contain pigments. They make it possible to improve the aesthetic effect by imparting greater interest to the face, the eyes and the lips and by heightening their color.

These products are covering products and in general have a temporary effect, that is to say they are easily removed using water or a solution containing surfactants.

Nowadays, there is increasing demand for permanent or semi-permanent coloring products, for example to impart to the skin a color close to that of a natural tan, to make up the nails more durably, to emphasise the contour of the eyes and of the lips in a more sustained manner and also for tattooing. The formulations used for this purpose in particular make it possible to look good and be made up for several days without having to make up every day.

In the sense of the present invention, “composition for permanent or semi-permanent coloring” is understood to mean any composition having a particular affinity for the skin, the eyelids, the lips or the nails enabling it to impart to the skin, the eyelids, the lips or the nails a coloration which is non-covering (which does not render opaque) and durable, which is not easily removed with water nor using a solvent and which resists both rubbing and washing with a solution containing surfactants. Such a durable coloration is thus distinct from the superficial, covering and temporary coloration imparted for example by a typical cosmetic product.

In the sense of the present invention, “coloring agent” is understood to mean any substance having a particular affinity for the skin, the eyelids, the lips or the nails enabling it to impart to the skin, the eyelids, the lips or the nails a durable coloration such as that defined above.

However, certain coloring agents have the disadvantage of being unstable towards water, oxidising agents or light, that is to say they undergo degradation by a hydrolysis, oxidation, photolysis or photodegradation mechanism. The phenomenon of oxidation can have several causes, in particular the presence of water, of oxidising agents such as the oxygen in the air, or light and in particular ultraviolet radiation.

These coloring agents can also have the disadvantage of reacting with other cosmetic active agents currently used in cosmetics, when they are used in aqueous solution.

Further, the compositions for permanent or semi-permanent coloring of the skin, the eyelids, the lips or the nails, in the sense of the present invention, are generally aqueous compositions in which the coloring agents must be dissolved. The lack of solubility of these components diminishes the coloring power of these compositions. This criterion of solubility can reduce the number of coloring agents that can be used for the coloring of the skin, the eyelids, the lips or the nails. This is particularly the case for compounds of high melting point.

### DETAILED DESCRIPTION OF THE PREFERRED EMBODIMENTS

It has now surprisingly been discovered that by using a new process for preparation of a composition for permanent or semi-permanent coloring of the skin, the eyelids, the lips or the nails, it is possible in a very short time, for example less than 2 minutes, to obtain compositions with a higher or lower concentration of coloring agent(s) as required, in particular without preservative, making it possible to overcome the disadvantages described above.

This process may be simply implemented and is suited to the needs of the consumer. A fluid under pressure, whose temperature is preferably greater than or equal to 30° C., is passed for a very short time period through at least one coloring agent in solid or paste form, preferably solid, and still more preferably in powder form.

It thus makes it possible to use in anhydrous form coloring agents unstable in aqueous compositions, either because they react with water or with the ambient humidity, or because they react in aqueous solution with compounds which do not react with them in an anhydrous composition, and to store them without risk of degradation.

The compositions prepared according to this process can display limited storage stability, which is not a disadvantage here since the process results in a ready-to-use composition, intended to be used rapidly after its preparation, for example in the 5 minutes following its preparation, in particular after cooling to a temperature acceptable for keratinic materials, preferably less than 60° C., better less than 50° C. The composition can also be used up to one week or more after its preparation, depending on the rate of degradation of the coloring agent used.

Given the very short preparation time, the compositions for permanent or semi-permanent coloring of the skin, the eyelids, the lips or the nails can be prepared “on demand” by mixing the coloring agents, i.e. depending on the color sought, in one particular embodiment.

According to another embodiment, since the coloring agents can be packed in a ready-to-use device, it is not necessary to determine the concentrations of the agents in solution beforehand, which limits measurement errors due to the user.

In addition, the process according to the invention makes it possible to avoid the use of multi-compartment bottles, which makes the process particularly economical and more reliable for the user.

The composition thus obtained can be used alone or mixed with another composition.

Thus an object of the invention is a process for preparation of a composition useful, e.g., for permanent or semi-permanent coloring of the skin, the eyelids, the lips or the nails, etc., comprising a step of percolation of a fluid under a pressure of at least 3 bars (3×105 Pa) through at least one coloring agent in solid or paste form.

Another object of the invention is the composition obtained by the process according to the invention.

Also an object of the invention is the use of the composition obtained according to the process of the invention for the permanent or semi-permanent coloring of the skin, the eyelids, mucous membranes, the lips or the nails and preferably of the skin, the eyelids or the lips.

Another object of the invention is a process for permanent or semi-permanent coloring of the skin, the eyelids, the lips or the nails using the composition for permanent or semi-permanent coloring of the skin, the eyelids, the lips or the nails obtained according to the process of the invention.

A further object of the invention is a packaging device making it possible to implement the preparation process of the present invention.

Other objects, characteristics, aspects and advantages of the invention will appear still more clearly on the reading of the further nonlimiting description and the examples which follows.

According to the invention, the process for preparation of a composition useful for, e.g., permanent or semi-permanent coloring of the skin, the eyelids, the lips or the nails, comprises percolation of a fluid preferably at a temperature greater than or equal to 30° C., still better ranging from 30 to 150° C. and still more preferably from 40 to 120° C., under a pressure of at least 3 bars (3×105 Pa) through a coloring agent in solid or paste form.

Percolation is a movement of fluid through a porous medium allowing the passage of the fluid, under the action or the effect of pressure.

The fluid can comprise water vapour (e.g., steam) optionally accompanied by liquid water, and/or by one or several cosmetically acceptable liquid and/or gaseous solvents. Preferably, the fluid contains at least water vapour which can be accompanied by liquid water, and still more preferably it is water vapour/steam which can be accompanied by liquid water.

As the organic solvent, C1-C4 lower alcohols such as ethanol and isopropanol, polyols and polyol ethers such as 2-butoxyethanol, propylene glycol, propylene glycol monomethyl ether, diethylene glycol monoethyl ether and diethylene glycol monomethyl ether, and also aromatic alcohols such as benzyl alcohol or phenoxyethanol, and mixtures thereof, can for example be mentioned.

The coloring agent is in solid or paste form, preferably in solid form, and still more preferably in powder form.

“Paste form” in the sense of the present invention is understood to mean a consistency intermediate between a solid phase and a liquid phase. The viscosity of this paste phase is preferably greater than or equal to 0.1 Pa.sec, and still more preferably greater than or equal to 1 Pa.sec, this being at 25° C. with a shear rate of 10 sec-1.

The process of the present invention can be implemented on the basis of a standard device making it possible to generate a fluid under pressure, at a temperature preferably greater than or equal to 30° C., still better ranging from 30 to 150° C., and still more preferably from 40 to 120° C. Such a device includes a pressure-resistant chamber, equipped with a heating block, and a circuit for directing the fluid produced towards the coloring agent used in the invention.

According to another embodiment, the device further includes a tank for liquid(s) and a pump allowing the directing of the liquid or liquids into the chamber.

The liquid contained in the tank is preferably either water, or a cosmetically acceptable solvent or a mixture of several cosmetically acceptable solvents, or else a mixture of water and of one or several cosmetically acceptable solvents. Preferably, the liquid contains at least water and still more preferably it is water.

A particularly useful device for the implementation of the process of the present invention is a coffee machine of the “espresso” type. Such machines are well known in the art. For example, these machines are described in the patents AT 168405, U.S. Pat. No. 2,688,911, DE 32433870 and IT 1265636.

According to a particular embodiment of the invention, the percolation step is implemented with a fluid at a temperature preferably greater than or equal to 30° C., still better ranging from 30 to 150° C., under a pressure of 3 to 30 bars (3×10⁵ to 3×106 Pa), preferably at least 4 bars (4×105 Pa), more preferably greater than or equal to 10 bars (106 Pa) and quite particularly from 10 to 30 bars (106 to 3×106 Pa).

A cosmetic composition containing at least one coloring agent in liquid or paste form can be used directly in the device generating the fluid under pressure in a container intended for that use. It can also be packed in a special packaging device containing a closed housing bounded by at least one wall permeable at least in part to the fluid under a pressure of at least 3 bars (3×105 Pa). Such devices are for example described in the patent applications WO 00/56629, EP 512470, U.S. Pat. No. 5,897,899 or WO 99/03573. These packaging devices are generally impermeable to air, moisture and/or light.

According to one particular embodiment, the housing is bounded by two sealed films. According to another embodiment, the housing is bounded by a tray closed by a lid.

These devices can be manufactured from woven or non-woven materials, of plastic or plant matter, for example of cellulose, or of a metal such as aluminium or of a composite. Such devices are for example described in the patent applications WO 00/56629, EP 512470, U.S. Pat. No. 5,897,899 or WO 99/03573.

The coloring agents used in the invention are in particular preferably selected from the self-tanning agents, indole compounds, coloring plant extracts, flavylium salts not substituted in the 3 position and substituted with at least one hydroxy or alkoxy group, dye precursors of the polyphenol type, natural dyes, cochineal carmine, fluoranes or alkali metal salts thereof, melanogenesis activators, and mixtures thereof.

As self-tanning agents, in particular mono- or polycarbonyl compounds can be mentioned. The mono- or polycarbonyl self-tanning agents can for example be selected from isatin, alloxan, ninhydrin, glyceraldehyde, mesotartaric aldehyde, glutaraldehyde, erythrulose, derivatives of pyrazolin-4,5-diones as described in the patent application FR 2466492 and WO 97/35842, dihydroxyacetone (DHA), and derivatives of 4,4-dihydroxy-pyrazoline-5-ones as described in the patent application EP 0903342.

In a particularly preferred embodiment of the invention, dihydroxyacetone (DHA) will more particularly be used as the self-tanning agent.

As indole compounds, in particular the monohydroxyindoles as described in the patent FR 2651126 or the dihydroxyindoles as described in the patent EP 425324 can be mentioned. Particular examples of these compounds are 4-, 5-, 6- or 7-hydroxyindoles, 5,6-dihydroxyindole, 2-methyl-5,6-dihydroxyindole, 3-methyl-5,6-dihydroxyindole and 2,3-dimethyl-5,6-dihydroxyindole, and more preferably the 5- and 6-hydroxyindoles and 5,6-dihydroxyindole, and still more preferably 5,6-dihydroxyindole.

As coloring plant extracts, the following can for example be mentioned:
plant extracts of the genus Sorgho such as Sorghum caudatum and Sorghum Bicolor as described in the applications EP 1172090 and EP 1327437.

The flavylium salts not substituted in position 3 and substituted with at least one hydroxy or alkoxy group are obtained by synthesis or from a plant extract containing them or else from a concentrated extract. These salts are in particular described in the application EP 1172090 and preferred examples of these salts are the chlorides of the following compounds:
4′,5,7-trihydroxyflavylium, commonly called “apigeninidine chloride”,
3′,4′,7-trihydroxyflavylium,
4′-hydroxyflavylium,
4′,7-dihydroxyflavylium,
3′,4′-dihydroxyflavylium,
3′,4′-dihydroxy-7-methoxy-flavylium,
3′,4′,5,7-tetrahydroxyflavylium, and
3′,4′,5′,5,7-pentahydroxyflavylium.

Among these compounds, apigeninidine chloride (4′,5,7-trihydroxyflavylium chloride) and 3′,4′,7-trihydroxyflavylium chloride are still more particularly preferred.

Apigeninidine chloride can also be used in the form of a plant extract, easily prepared by extraction, and isolated, from leaves of Sorghum caudatum by the processes described in the patents CN 1065284A and CN 1035512C or any other modifications of these processes.

As dye precursors of the polyphenol type (i.e. ortho-diphenol), those described in the application EP 1229892 can be used in the process of the invention. The preferred dye precursors of the polyphenol type are:
flavanols such as catechin and epicatechin gallate
flavonols such as quercetin,
anthocyanidines such as peonidin,
anthocyanines, for example oenin,
hydroxybenzoates, for example gallic acid,
flavones such as luteolin, and
iridoids such as oleuropein,
and these products can be osylated (for example glucosylated) and/or in the form of oligomers (procyanidines);
hydroxystilbenes, for example 3,3′,4,5′-tetrahydroxystilbene, optionally osylated (for example glucosylated);
3,4-dihydroxyphenylalanine and derivatives thereof,
2,3-dihydroxyphenylalanine and derivatives thereof,
4,5-dihydroxyphenylalanine and derivatives thereof,
dihydroxycinnamates such as caffeic acid and chlorogenic acid,
hydroxycoumarins,
hydroxyisocoumarins,
hydroxycoumarones,
hydroxyisocoumarones,
hydroxychalcones,
hydroxychromones,
anthocyans,
quinones, and
hydroxyxanthones.

As natural dyes, for example:
the flavonoids and esters, ethers, heterosides and polymers thereof,
non-flavonoid polyphenols and esters, ethers, heterosides and polymers thereof,
carotenoids such as the β-carotenes, and the β-laines,
chlorophylls,
carbohydrates (caramel), and
melanins, for example eumelanins and pheomelanins, can be mentioned for example.

The cochineal carmine usable in the process of the invention is that in particular described in the application WO 02/41867. It is also known in particular under the following names: CI 75470, Natural Red 4, Carminic Acid, Carmine 5297, Carmine Ultra-Fine, Carminic Acid Lake and E 120.

The red or orange dyes usable in the process of the invention are in particular selected from the fluoranes and alkali metal salts thereof as described in the application FR 2840806. More particularly, tetrabromofluorescein or eosin known under the name CTFA CI 45380 or Red 21, phloxine B known under the name CTFA CI 45410 or Red 27, diiodo-fluorescein known under the name CTFA CI 45425 or Orange 10, dibromofluorescein known under the name CTFA CI 45370 or Orange 5, the sodium salt of tetrabromofluorescein known under the name CTFA CI 45380 (Na salt) or Red 22, the sodium salt of phloxine B known under the name CTFA CI 45410 (Na salt) or Red 28, the sodium salt of diiodo-fluorescein known under the name CTFA CI 45425 (Na salt) or Orange 11, erythrosine known under the name CTFA CI 45430 or Acid Red 51 and phloxine known under the name CTFA CI 45405 or Acid Red 98 can be used.

As activators of melanogenesis, those described in the applications EP 1385472 and FR 2831439 can in particular be mentioned, and more particularly analogues of substrates of tyrosinase, a key enzyme in melanogenesis, such as tyrosine, L-dopa or L-dihydroxyphenyl-alanine, activators of the activity or expression of tyrosinase such as forskoline, xanthine bases (theophylline, caffeine), pro-opiomelanocortic peptides (ACTH, alpha-MSH or other agonists of MCI receptors), diacylglycerols, aliphatic or cyclic diols, psoralenes, prostaglandins and analogues, activators of NO/cGMP dependent protein kinase G, activators of transfer of melanosomes towards the keratinocytes such as serine proteases or agonists of the PAR-2 receptors, or else an extract of Burnet (Sanguisorba officinalis) or an extract of at least one plant of the Chrysanthemum genus, particularly of the species Chrysanthemum sinensis.

The coloring agent or agents can be used in the process of the invention mixed with one or several adjuvants in solid or paste and preferably powder form. The adjuvants can be selected from clays, salts, anionic, nonionic, cationic or zwitterionic surfactants, natural or synthetic thickeners, optionally modified starch, glass beads, silica, nylon, alumina, titanium dioxide, zeolites, poly(methyl methacrylate) (PMMA), chitosan, maltodextrin, cyclodextrin, mono- or disaccharides such as glucose, sucrose, sorbitol or fructose, zinc oxide, zirconium oxide, silicabades, talc, polyaspartic acid, borosilicates in particular of calcium, polyethylene, cotton, polytetrafluoroethylene (PTFE), cellulose and derivatives thereof, superabsorbent compounds, and magnesium or calcium carbonates, maize grains, polyacrylamide, porous hydroxyapatite, silk, collagen, wood sawdust, fucus powder, flours or extracts of wheat, rice, peas, lupin, soya or barley, crosslinked polyvinylpyrrolidone, calcium alginate, activated carbon, particles of poly(vinylidene chloride/acrylonitrile), in particular those marketed under the general name of “Expancel®” by the company AKZO NOBEL under the specific names “Expancel® WE” or “DE” Expancels and mixtures thereof.

When one or several adjuvants are present, the coloring agent or agents used in the invention are preferably present in a quantity ranging from 0.5 to 99% by weight, still better from 1 to 80% by weight, and still more preferably from 2 to 60% by weight relative to the total weight of coloring agent(s) and additive(s) in solid or paste form.

When plants or plant extracts are used in the process of the present invention, they may be pretreated before the percolation step. The pretreatment can be drying, roasting, cryogrinding or freeze-drying.

The composition for permanent or semi-permanent coloring obtained according to the process of the invention, apart from the coloring agent or agents and the component(s) of the fluid, namely water and/or cosmetically acceptable solvent(s), optionally contains all or part of the adjuvant(s) present in the mixture in solid or paste form.

The invention also concerns a composition obtainable by the process according to the invention, the particularly preferred composition being free from preservatives.

From the preparation process of the invention, a composition for permanent or semi-permanent coloring of, e.g., the skin, the eyelids, the lips or the nails, preferably of the skin, the eyelids or the lips, is obtained, which can be applied directly onto the skin, the eyelids, the lips or the nails, preferably onto the skin, the eyelids or the lips, or which can be mixed with a medium appropriate for the coloring operation, or else at least one additive typically used in cosmetics can be added to it by an operator. Also, at least two compositions obtained by the process of the invention can be mixed. The composition for coloring the skin, the eyelids, the lips or the nails optionally resulting from mixture(s) and/or addition(s) mentioned above will hereinafter be referred to as final coloring composition or final composition.

One particular embodiment of the invention comprises applying the composition obtained, in particular to the skin, by means of a device not necessitating human intervention and optionally equipped with a means of cooling.

Another particular embodiment comprises ingesting the composition for permanent or semi-permanent coloring obtained according to the process of the invention, preferentially, of course, when no toxicity problem is known in the art, for example when the coloring agent is a carotenoid, for example β-carotene.

The quantity of the coloring agent or agents present in the final coloring composition in general preferably lies in the range from 0.001 to 50% by weight approximately, preferably from 0.005 to 30% by weight and still more preferably from 0.01 to 20% by weight relative to the total weight of the final coloring composition.

In the case where the composition for permanent or semi-permanent coloring of the skin, the eyelids, the lips or the nails obtained by the process of the present invention is mixed with a medium appropriate for the coloring operation, the medium generally comprises water or a mixture of water and at least one organic solvent to solubilize the compounds which would not be sufficiently soluble in water. As organic solvents, for example C1-C4 lower alcohols such as ethanol and isopropanol, polyols and polyol ethers such as 2-butoxyethanol, propylene glycol, propylene glycol monomethyl ether, diethylene glycol monoethyl ether and diethylene glycol monomethyl ether, and aromatic alcohols such as benzyl alcohol or phenoxyethanol, and mixtures thereof can be mentioned.

The solvents are preferably present in proportions preferably lying between 1 and 40% by weight relative to the total weight of the final composition, and still more preferably between 5 and 30% by weight.

At least one additive typically used in cosmetics can also be added to the compositions for permanent or semi-permanent coloring of the skin, the eyelids, the lips or the nails obtained according to the process of the present invention. As examples of such additives, anionic, cationic, nonionic, amphoteric or zwitterionic surfactants or mixtures thereof; anionic, cationic, nonionic, amphoteric or zwitterionic polymers or mixtures thereof; inorganic or organic thickening agents, and in particular anionic, cationic, nonionic and amphoteric polymeric associative thickeners; antioxidants; penetration agents; sequestering agents; perfumes; buffers; dispersants; conditioning agents; film-forming agents; ceramides; preservatives and opacifiers can be mentioned.

The above additives are in general present in a quantity which for each of them lies between 0.01 and 20% by weight relative to the weight of the final composition.

Of course, the person skilled in the art will take care to select this or these optional compounds in such a manner that the advantageous properties intrinsically attached to the coloring composition according to the invention are not, or not substantially, adversely affected by the addition or additions envisaged.

The pH of the final composition generally lies between 3 and 12, and preferably between 5 and 11. It can be adjusted to the desired value by means of acidification or basification agents normally used in cosmetics or else by means of typical buffer systems.

Among these acidification agents, by way of example, inorganic or organic acids such as hydrochloric acid, orthophosphoric acid, sulphuric acid, and carboxylic acids such as acetic acid, tartaric acid, citric acid, lactic acid and the sulphonic acids can be mentioned.

Among the basifying salts, by way of example, ammonia, alkali metal carbonates, alkanolamines such as mono-, di- and triethanolamine and derivatives thereof, sodium or potassium hydroxides and compounds of the following formula: 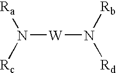
wherein W is a propylene residue optionally substituted with a hydroxyl group or a C1-C4 alkyl group, and Ra, Rb, Rc and Rd are the same or different and represent a hydrogen atom, or a C1-C4 alkyl or C1-C4 hydroxyalkyl group can be mentioned.

The final composition for coloring of the skin, the eyelids, the lips or the nails can be in various forms, such as in the form of liquids, creams, gels or in any other form appropriate for effecting coloring of the skin, the eyelids, the lips or the nails.

The present invention also concerns a process for permanent or semi-permanent coloring of, e.g., the skin, the eyelids, the lips or the nails, comprising the preparation of a composition for coloring of the skin, the eyelids, the lips or the nails by the process according to the invention, and its application onto the skin, the eyelids, the lips or the nails, for example by an operator or by a device not necessitating human intervention. The duration of application can vary between 15 seconds and 1 hour.

Before application, the composition for permanent or semi-permanent coloring obtained according to the process of the invention can be mixed with a medium appropriate for the coloring operation and/or with one or several additives typically used in cosmetics such as are described above.

Another embodiment of the invention is preparing at least two compositions for permanent or semi-permanent coloring according to the process of the invention, mixing them, and optionally adding a medium appropriate for the coloring operation and/or one or several additives typically used in cosmetics such as are described above, then applying the final composition obtained onto the skin, the eyelids, the lips or the nails.

The examples below are intended to illustrate the present invention, but not limit it.

### EXAMPLE 1

The following ingredients, in powder form, are mixed:

| | |
|---|---|
| Dihydroxyacetone | 50% by weight |
| Maltodextrin | 50% by weight |

relative to the total weight of the powder mixture.

This mixture, which is in a container intended to accommodate a solid compound, is placed in a commercial espresso machine. The water vapour produced by the machine then passes through this powder mixture. In this way, a self-tanning composition ready to be applied onto the skin is obtained.

### EXAMPLE 2

According to the same mode of operation as in Example 1, water vapour is passed onto a dihydroxyacetone powder which is in the container intended to accommodate a solid compound. In this way, a self-tanning composition ready to be applied onto the skin is obtained.

The above written description of the invention provides a manner and process of making and using it such that any person skilled in this art is enabled to make and use the same, this enablement being provided in particular for the subject matter of the appended claims, which make up a part of the original description and including a process for preparing a (cosmetic) composition useful for treating keratin materials and/or mucous membranes, wherein it comprises a step of percolating a fluid under a pressure of at least 3 bars through at least one coloring agent, in solid or pasty form, the composition prepared, and a process using the composition for the permanent or semi-permanent coloring of the skin, the eyelids, the lips or the nails, for example.

As used herein, the phrases “selected from the group consisting of,” “chosen from,” “selected from,” and the like include mixtures of the specified materials.

Where compounds are described as, e.g, “cellulose and its derivatives” and the like an alternate is “cellulose and cellulose compounds” where cellulose derivatives and compounds share a common core/structure with cellulose. One of ordinary skill in the art knows how to identify derivatives and compounds of compound X based on the structure of X and the similarity in structure of the derivatives and compounds.

Where a term is enclosed by parentheses it is an optional modifier. For example, the term “(cosmetic) composition” describes both a composition in general and a cosmetic composition.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid under a pressure of at least 3 bars through at least one coloring agent in solid or paste form.

2. A process according to claim 1, wherein the fluid comprises water vapour optionally accompanied by liquid water and/or by one or several cosmetically acceptable liquid and/or gaseous solvents.

3. A process according to claim 2, wherein the fluid comprises liquid water.

4. A process according to claim 1, wherein the coloring agent is selected from self-tanning agents, indole compounds, coloring plant extracts, flavylium salts not substituted in the 3 position and substituted with at least one hydroxy or alkoxy group, dye precursors of the polyphenol type, natural dyes, cochineal carmine, fluoranes or alkali metal salts thereof, melanogenesis activators, and mixtures thereof.

5. A process according to claim 1, wherein the wherein the coloring agent comprises at least one mono- or polycarbonyl compound.

6. A process according to claim 1, wherein the wherein the coloring agent comprises at least one selected from isatin, alloxan, ninhydrin, glyceraldehyde, mesotartaric aldehyde, glutaraldehyde, erythrulose, derivatives of pyrazolin-4,5-diones, dihydroxyacetone (DHA), derivatives of 4,4-dihydroxypyrazoline-5-ones, and mixtures thereof.

7. A process according to claim 1, wherein the wherein the coloring agent comprises dihydroxyacetone.

8. A process according to claim 1, wherein the wherein the coloring agent comprises at least one selected from monohydroxyindoles and dihydroxyindoles.

9. A process according to claim 8, wherein the wherein the coloring agent comprises 5,6-dihydroxyindole.

10. A process according to claim 1, wherein the wherein the coloring agent comprises at least one plant extract selected from the plant extracts of the genus Sorgho.

11. A process according to claim 1, wherein the wherein the coloring agent comprises at least one plant extract selected from Sorghum caudatum and Sorghum Bicolor.

12. A process according to claim 1, wherein the wherein the coloring agent comprises at least one selected from apigeninidine chloride and 3′,4′,7-trihydroxyflavylium chloride.

13. A process according to claim 1, wherein the wherein the coloring agent comprises at least one orthodiphenol.

14. A process according to claim 1, wherein the wherein the coloring agent comprises at least one selected from flavonoids and esters, ethers, heterosides and polymers thereof, non-flavonoid polyphenols and esters, ethers, heterosides and polymers thereof, carotenoids and β-laines, chlorophylls, carbohydrates and melanins.

15. A process according to claim 1, wherein the coloring agent is present as a mixture with at least one adjuvant.

16. A process according to claim 15, wherein the adjuvant is selected from clays, salts, anionic, nonionic, cationic or zwitterionic surfactants, natural or synthetic thickeners, optionally modified starch, glass beads, silica, nylon, alumina, titanium dioxide, zeolites, poly(methyl methacrylate) (PMMA), chitosan, maltodextrin, cyclodextrin, mono- or disaccharides, zinc oxide, zirconium oxide, silicabades, talc, polyaspartic acid, borosilicates, polyethylene, cotton, polytetrafluoroethylene (PTFE), cellulose and derivatives thereof, superabsorbent compounds, magnesium or calcium carbonates, maize grains, polyacrylamide, porous hydroxyapatite, silk, collagen, wood sawdust, fucus powder, flours or extracts of wheat, rice, peas, lupin, soya or barley, crosslinked polyvinylpyrrolidone, calcium alginate, activated carbon, particles of poly(vinylidene chloride/acrylonitrile), and mixtures thereof.

17. A process according to claim 15, wherein the coloring agent is present in 0.5-99% by weight relative to the total weight of coloring agent(s) and adjuvant(s) in solid or paste form.

18. A process according to claim 1, wherein the fluid is under a pressure of 3 to 30 bars.

19. A process according to claim 18, wherein the fluid is under a pressure of at least 10 bars.

20. A composition prepared by a process comprising percolating a fluid under a pressure of at least 3 bars through at least one coloring agent in solid or paste form.

21. A composition according to claim 20, wherein said composition does not comprise any preservative.

22. A composition according to claim 20, prepared by a process comprising comprising percolating a fluid under a pressure of at least 3 bars through at least one coloring agent in solid or paste form, the coloring agent being present in a mixture with at least one adjuvant, the composition comprising all or part of the adjuvant(s) present in the mixture in solid or paste form.

23. A process for the permanent or semi-permanent coloring of the skin, the eyelids, the lips or the nails, comprising applying onto the skin, the eyelids, the lips or the nails one or more compositions according to claim 20.

24. A device for packaging a composition, comprising a closed housing delimited by at least one wall that is at least partially permeable to fluid at a pressure of at least 3 bar, the device having therein a composition comprising at least one coloring agent, in solid or pasty form, the device housing being optionally bounded by two sealed films or by a tray closed by a lid.
